SEQUENCE LISTING (3 sequences)

SEQ ID NO: 1 - protein, 859 aa, Homo sapiens
MACLHETRTPSPSFGGFVSTLSEASMRKLDPDTSDCTPEKDLTPTHVLQLHEQDAGGPGGAAGSPESRASRVRADEVRLQCQSGSGFLEGLFGCLRPVWTMIGKAYSTEHKQQQEDLWEVPFEEILDLQWVGSGAQGAVFLGRFHGEEVAVKKVRDLKETDIKHLRKLKHPNIITFKGVCTQAPCYCILMEFCAQGQLYEVLRAGRPVTPSLLVDWSMGIAGGMNYLHLHKIIHRDLKSPNMLITYDDVVKISDFGTSKELSDKSTKMSFAGTVAWMAPEVIRNEPVSEKVDIWSFGVVLWELLTGEIPYKDVDSSAIIWGVGSNSLHLPVPSSCPDGFKILLRQCWNSKPRNRPSFRQILLHLDIASADVLSTPQETYFKSQAEWREEVKLHFEKIKSEGTCLHRLEEELVMRRREELRHALDIREHYERKLERANNLYMELNALMLQLELKERELLRREQALERRCPGLLKPHPSRGLLHGNTMEKLIKKRNVPQKLSPHSKRPDILKTESLLPKLDAALSGVGLPGCPKGPPSPGRSRRGKTRHRKASAKGSCGDLPGLRTAVPPHEPGGPGSPGGLGGGPSAWEACPPALRGLHHDLLLRKMSSSSPDLLSAALGSRGRGATGGAGDPGSPPPARGDTPPSEGSAPGSTSPDSPGGAKGEPPPPVGPGEGVGLLGTGREGTSGRGGSRAGSQHLTPAALLYRAAVTRSQKRGISSEEEEGEVDSEVELTSSQRWPQSLNMRQSLSTFSSENPSDGEEGTASEPSPSGTPEVGSTNTDERPDERSDDMCSQGSEIPLDPPPSEVIPGPEPSSLPIPHQELLRERGPPNSEDSDCDSTELDNSNSVDALRPPASLPP

SEQ ID NO: 2 - protein, 892 aa, Homo sapiens
MACLHETRTPSPSFGGFVSTLSEASMRKLDPDTSDCTPEKDLTPTQCVLRDVVPLGGQGGGGPSPSPGGEPPPEPFANSVLQLHEQDAGGPGGAAGSPESRASRVRADEVRLQCQSGSGFLEGLFGCLRPVWTMIGKAYSTEHKQQQEDLWEVPFEEILDLQWVGSGAQGAVFLGRFHGEEVAVKKVRDLKETDIKHLRKLKHPNIITFKGVCTQAPCYCILMEFCAQGQLYEVLRAGRPVTPSLLVDWSMGIAGGMNYLHLHKIIHRDLKSPNMLITYDDVVKISDFGTSKELSDKSTKMSFAGTVAWMAPEVIRNEPVSEKVDIWSFGVVLWELLTGEIPYKDVDSSAIIWGVGSNSLHLPVPSSCPDGFKILLRQCWNSKPRNRPSFRQILLHLDIASADVLSTPQETYFKSQAEWREEVKLHFEKIKSEGTCLHRLEEELVMRRREELRHALDIREHYERKLERANNLYMELNALMLQLELKERELLRREQALERRCPGLLKPHPSRGLLHGNTMEKLIKKRNVPQKLSPHSKRPDILKTESLLPKLDAALSGVGLPGCPKGPPSPGRSRRGKTRHRKASAKGSCGDLPGLRTAVPPHEPGGPGSPGGLGGGPSAWEACPPALRGLHHDLLLRKMSSSSPDLLSAALGSRGRGATGGAGDPGSPPPARGDTPPSEGSAPGSTSPDSPGGAKGEPPPPVGPGEGVGLLGTGREGTSGRGGSRAGSQHLTPAALLYRAAVTRSQKRGISSEEEEGEVDSEVELTSSQRWPQSLNMRQSLSTFSSENPSDGEEGTASEPSPSGTPEVGSTNTDERPDERSDDMCSQGSEIPLDPPPSEVIPGPEPSSLPIPHQELLRERGPPNSEDSDCDSTELDNSNSVDALRPPASLPP

SEQ ID NO: 3 - protein, 34 aa, Homo sapiens
QCVLRDVVPLGGQGGGGPSPSPGGEPPPEPFANS

(19) United States
(12) Patent Application Publication
(10) Pub. No.: US 2025/0041451 A1
(43) Pub. Date: Feb. 6, 2025

(54) GENE THERAPY FOR HEARING LOSS

(71) Applicant: Perceive Biotherapeutics, Inc., Brisbane, CA (US)

(72) Inventor: Derek Welsbie, Rancho Santa Fe, CA (US)

(21) Appl. No.: 18/554,160

(22) PCT Filed: Apr. 6, 2022
(86) PCT No.: PCT/US2022/023699

§ 371 Date: Oct. 5, 2023

Related U.S. Application Data
(60) Provisional application No. 63/171,498, filed on Apr. 6, 2021.
(60) Provisional application No. 63/295,364, filed on Dec. 30, 2021.

(51) Int. Cl. A61K 48/00; A61P 27/16; C12N 15/86

(52) U.S. Cl. CPC A61K 48/0058; A61P 27/16; C12N 15/86; C12N 2750/14143; C12N 2750/14145; C12N 2750/14171

(57) ABSTRACT

The disclosure provides compositions and methods for the treatment of hearing loss disorder.

### CROSS-REFERENCE TO RELATED APPLICATIONS

This application claims priority benefit of U.S. provisional application No. 63/171,498 filed Apr. 6, 2021 and U.S. provisional application No. 63/295,364, filed Dec. 30, 2021; each of which is incorporated by reference for all purposes.

### BACKGROUND OF THE INVENTION

Hearing loss is one of the most common disabilities affecting the world's population today. According to the National Health and Nutritional Examination Survey, nearly two thirds of U.S. adults aged 70 years and older are affected by hearing loss. Hearing loss is currently treated by rehabilitation methods, for example, conventional hearing aids. For more severe forms, cochlear implants are often used. Efforts are continuing to improve these devices to help users understand speech in noisy environments and to appreciate music. However, neither approach satisfactorily restores hearing sensitivity. Therefore new approaches for treatment or prevention of hearing loss are necessary.

### BRIEF SUMMARY OF THE INVENTION

Provided herein are compositions and methods for treating or preventing hearing loss in a subject. In some embodiments, the hearing loss is age-related hearing loss, hereditary hearing loss, chemotherapy induced hearing loss, aminoglycoside-induced hearing loss, trauma-induced hearing loss, disease-induced hearing loss, or noise-induced hearing loss.

In one aspect, the disclosure features a method for treating or preventing hearing loss in a subject comprise administering a dual-leucine zipper kinase (DLK) inhibitor to the subject. In some embodiments, the hearing loss is selected from the group consisting of age-related hearing loss, hereditary hearing loss, chemotherapy induced hearing loss, aminoglycoside-induced hearing loss, trauma-induced hearing loss, disease-induced hearing loss, and noise-induced hearing loss. In some embodiments, the hearing loss is the result of loss of auditory hair cells (HC). In some embodiments, the hearing loss is the result of loss of spiral ganglion cells (SG). In further embodiments, the subject has hearing loss following treatment with an ototoxic drug. In some embodiments, the DLK inhibitor is administered to a subject prior to treatment with an ototoxic drug. In some embodiments, the ototoxic drug is an aminoglycoside. In some embodiments, the inhibitor is a biologic inhibitor. In some embodiments, the inhibitor is a dominant negative form of a DLK polypeptide (dnDLK). In some embodiments, the dominant negative DLK polypeptide is administered by gene therapy, using a vector comprising a nucleic acid encoding a dominant negative form of the DLK polypeptide. In some embodiments, the dominant negative DLK polypeptide comprises a substitution K152A. In some embodiments, the dominant negative DLK polypeptide is a DLK leucine zipper domain, such as amino acids 372-487 of SEQ ID NO:1. In some embodiments, the dominant negative form of the DLK polypeptide comprises a substitution at position K391 and/or H393. In some embodiments, the dominant negative DLK polypeptide comprises a substitution K391D/E and/or H393D/E. In some embodiments, when the dominant negative polypeptide comprises a substitution at K391 and/or H393, such as K391D/E and/or H393D/E, the DLK further comprises at least one substitution at position K398, H405, L407, V412, R414, K453, L458, or R460. In some embodiments, the substitution is K398R, H405D/E, L407D/E, V412D/E, R414D/E, K453R, L458E/E, or R460D/E.

In some embodiments, a viral vector comprising a nucleic acid encoding the dominant negative form of the DLK polypeptide is administered to the subject. In some embodiments, the viral vector is an adeno-associated vector.

In some embodiments, the DLK inhibitor or the viral vector is administered to the Inner Ear Hair Cells or Outer Ear Hair Cells of the subject and/or outer ear cells of the subject. In some embodiments, the nucleic acid sequence encoding the DLK inhibitor is administered to the spiral ganglion neurons in the cochlea of the subject. In some embodiments, the DLK inhibitor or viral vector is administered to the spiral ganglion in the cochlea of the subject.

In some embodiments, the DLK inhibitor or viral vector is administered intravenously, intrathecally, intratympanically, via round window administration, via semicircular canal delivery, or via stapedotomy.

### BRIEF DESCRIPTION OF THE DRAWINGS

FIG. 1 provides illustrative images of cochlear explant sections analyzed in the EXAMPLES section.

FIG. 2 shows 3-D Z-stack reconstructions of imaged cochlear explant treatment groups.

FIG. 3 shows cochlear explant hair cell count results for cochlear explant treatment groups. Counts for Fragments 1, 2, 3, 4, 5, and 6 are shown left to right for each treatment group.

### DETAILED DESCRIPTION OF THE INVENTION

### Introduction

Dual leucine zipper kinase (DLK) (also known as mitogen activated protein kinase kinase kinase 12 (MAP3K12)) phosphorylates and activates mitogen kinase kinases 4 & 7 (MKK4/7)(also known as mitogen activated protein kinases 4 & 7 (MAP2K4 & MAP2K7)) which in turn phosphorylate and activate JUN N-terminal kinase 1-3 (JNK1-3)(also known as mitogen activated protein kinases 8-10 (MAPK8-10)). Ultimately, phosphorylated and activated JNKs phosphorylate a variety of effector molecules (as an example, as the name implies, JUN) which leads to cell death and degeneration in a wide variety of systems. While DLK has been shown to be a key upstream regulator of JNKs in the injured retina, there are many other MAP3Ks that can regulate JNKs in other settings. Indeed, the small kinase family that includes DLK, called mixed lineage kinases (MLKs), includes other MAP3Ks like leucine zipper kinase (LZK)/MAP3K13, MLK1/MAP3K9, MLK2/MAP3K10 and MLK3/MAP3K11. Moreover, there are MAP3Ks that activate JNK that are not members of the MLK family, including, but not limited to, apoptosis signal-regulating kinase (ASK1)/MAP3K5 and transforming growth factor-β-activated kinase 1 (TAK1)/MAP3K7.

In multiple animal models of hearing loss, there is evidence of hair cell (HC) apoptosis (Hu et al., Hearing Research, 166:62-71, 2002; Yang et al., Hearing Research, 196:69-76, 2004; Wang et al., Molecular Pharmacology 71:654-666, 2007 Nagashima et al., Neurochemistry International 59-812-810, 2011); and activation of the JNK signaling pathway (evidenced by JUN phosphorylation). Further, inhibition of JNK signaling has shown to be protective in various models of hearing loss, including vertebral artery occlusion, aminoglycoside-induced cochlear toxicity and noise-induced hearing loss. Importantly, this protection is robust, lasting and associated with functional preservation (e.g., Omotehara et al., Otology and Neurotology 32:1422-1427, 2011; Eshraghi et al., Hearing Research 226:168-177, 2007; Eshraghi et al., Otology and Neurotology 27:1083-1088, 2006; Coleman et al., Hearing Research 226:104-113, 2007; Wang et al., Molecular Pharmacology, 2007, supra; Wang et al., J Neuroscience 23:8596-8607, 2003). Indeed, there is mixed clinical data suggesting that JNK inhibition can even be protective in patients (Staecker et al., Otology and Neurotology, 40:584-594, 2019). Prior work has demonstrated that the group of MLKs could be the relevant MAP3Ks controlling JNK activation in the cochlea. MLKs are expressed in HCs (Liu et al., J Neuroscience 34:11085-11095, 2014; Cai et al., J Neuroscience 35:5870-5883, 2015; Scheffer et al., J Neuroscience 35:6366-6380, 2015; Li et al, Scientific Data 5: article number 180199, 2018) and non-selective MLK inhibition with the kinase inhibitor CEP-1347 has been shown to increase HC survival and preserve hearing in multiple animal models (Ylikoski et al., Hearing Research 163:71-81, 2002; Pirvola et al., J Neuroscience; 20: 43-50, 2000).

In the retina, we have shown that other MLKs (e.g. MLK1-3) have little effect on retinal ganglion cell (RGC) survival (Welsbie et al, Neuron 94:1142-1154, 2017). Instead, survival is dependent on the simultaneous inhibition of DLK and its closest homolog, LZK. This is indicative that DLK and dual DLK/LZK inhibition will be protective to the cochlea, in particular, playing a role in hair cell (HC) survival, spiral ganglion cell (SGC) survival and/or the intervening synapses, all of which have been implicated in age-related, toxin-related and noise-induced hearing loss. The Examples section in the present disclosure provides further indication that DLK and dual DLK/LZK inhibition can reduce hair cell loss in cochlea.

### Definitions

As used herein, “dual leucine zipper kinase” or “DLK” is a serine/threonine protein kinase that is a member of the mixed lineage kinase subfamily. DLK comprises an N-terminal domain, a catalytic domain, a leucine zipper domain comprising two leucine zippers, and a C-terminal domain. Illustrative human DLK polypeptide sequences are available under UniProtKB accession number 12852. Human DLK is encoded by the mitogen-activated protein kinase kinase kinase 12 gene (MAP3K12), which is cytogenetically localized to chromosome region 12q13.13. Two protein isoforms have been identified: isoform 1 (sequence provided in SEQ ID NO:1) and isoform 2 (sequence provided in SEQ ID NO:2).

“Leucine zipper kinase” (LZK) is an MAP3K family member structurally related to DLK. LZK comprises an N-terminal domain, a catalytic domain (“kinase domain”), a leucine zipper domain comprising two leucine zippers, and a C-terminal domain. Illustrative human LZK polypeptide sequences are available under UniProtKB accession number 043283. Human LZK is encoded by the mitogen-activated protein kinase kinase kinase 13 gene (MAP3K13), which is cytogenetically localized to chromosome region 3q27.2.

As used herein, an “inhibitor” can be a small molecule, a drug, a chemical, a polypeptide, an interfering RNA, an antisense molecule, an antibody, an aptamer, a morpholino, a triple helix molecule, an siRNA, a shRNA, an miRNA, an antisense RNA or a ribozyme, to name a few. In some embodiments, expression and/or activity of a protein, for example, DLK can be inhibited in a cell by modifying the genome of a cell using CRISPR/Cas9, zinc finger nuclease (ZNFs) and other gene editing techniques. See, for example, Li et al. “Applications of genome editing technology in the targeted therapy of human diseases: mechanisms, advances and prospects,” Signal Transduction and Targeted Therapy 5(1) (2020). Similarly, LZK can be inhibited by modifying the genome of the cell to inhibit LZK.

As used herein, “gene therapy vector” refers to virus-derived sequence elements used to introduce a polynucleotide construct into a cell.

As used herein, “a viral vector” or “recombinant viral vector” refers to a gene therapy vector used to deliver a polynucleotide construct to a cell. It is understood that the term viral vector encompasses recombinant vector particles or virions (i.e., viral particles comprising at least one capsid or envelope protein and an encapsidated recombinant viral vector) and recombinant vector plasmids.

As used herein, a “recombinant viral vector” refers to a viral vector, for example, an AAV or lentiviral vector, comprising a nucleic acid sequence that is not normally present in the viral vector (i.e., a polynucleotide heterologous to the viral vector). In general, the heterologous nucleic acid is flanked by at least one, and generally by two, AAV inverted terminal repeat sequences (ITRs) when the vector is an AAV vector. When the vector is a lentiviral vector, the heterologous nucleic acid is flanked by at least one, and generally by two, long terminal repeat sequences (LTRs).

As used throughout, the term “nucleic acid” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

In some embodiments, a nucleic acid sequence encoding a DLK inhibitor, i.e., a polypeptide that inhibits DLK activity, is operably linked to a constitutive promoter. In other embodiments, a nucleic acid sequence encoding a DLK inhibitor is operably linked to an inducible promoter. In some instances, a nucleic acid sequence encoding a DLK inhibitor is operably linked to a tissue-specific or cell type-specific regulatory element. For example, in some instances, a nucleic acid sequence encoding an expression product of interest is operably linked to an inner ear hair cell-specific regulatory element e.g., a regulatory element that confers selective expression of the operably linked nucleic acid in an inner ear hair cell. See, for example, Boeda and Petit “A specific promoter of the sensory cells of the inner ear defined by transgenesis” Hum Mol. Genet. 19(15): 1581-9 (2001), for expression of a gene product under the control of the MYO7A promoter in inner ear hair cells. As used herein, specific expression does not mean that the expression product is expressed only in a specific tissue(s) or cell type(s), but refers to expression substantially limited to specific tissue(s) or cell types(s).

“Polypeptide,” “peptide,” and “protein” are used interchangeably herein to refer to a polymer of amino acid residues. All three terms apply to amino acid polymers in which one or more amino acid residue is an artificial chemical mimetic of a corresponding naturally occurring amino acid, as well as to naturally occurring amino acid polymers and non-naturally occurring amino acid polymers. As used herein, the terms encompass full-length proteins, truncated proteins, and fragments thereof, and amino acid chains, wherein the amino acid residues are linked by covalent peptide bonds.

As used herein, a “biologic” is a therapeutic agent that contains a polypeptide or polynucleotide.

As used herein, “hearing loss” or “hearing impairment” refers to a decrease in the ability to hear sounds, and includes are forms of hearing loss, including but not limited to, age-related hearing loss, hereditary hearing loss, chemotherapy induced hearing loss, aminoglycoside-induced hearing loss, trauma-induced hearing loss, disease-induced hearing loss, and noise-induced hearing loss. In some embodiments, hearing loss results from or is characterized by cell death. In some cases hearing loss that results from or is characterized by death of hair cells (HC) is treated. In some embodiments, hearing loss that results from or is characterized by death of spiral ganglion cells (SG) is treated. In some embodiments, hearing loss is characterized by both hair cell death and spiral ganglion cell death, and synaptic disruption.

The following description recites various aspects and embodiments of the present compositions and methods. No particular embodiment is intended to define the scope of the compositions and methods. Rather, the embodiments merely provide non-limiting examples of various compositions and methods that are at least included within the scope of the disclosed compositions and methods. The description is to be read from the perspective of one of ordinary skill in the art; therefore, information well known to the skilled artisan is not necessarily included.

### Compositions

Provided herein are DLK inhibitors and nucleic acid constructs comprising a nucleic acid encoding a DLK inhibitor. DLK inhibitors that improve neuron survival when introduced into neurons are known (see, e.g., Chen et al., J Neurosci 28:672-80, 20081; Nihalani et al., J. Biol. Chem. 275: 7273-7279, 2000; and Chalberg, WO2020/168111, each of which is incorporated by reference).

In some embodiments, the nucleic acid construct comprises a nucleic acid sequence encoding a dominant negative form of DLK. In the present application, human DLK substitution is designated as follows: the amino acid present in the reference sequence immediately precedes the position number and the amino acid that is substituted immediately follows the position number. Thus, for example, K152A means that K at position 152 of the reference sequence is substituted with A; and a substitution designated as K391D/E means that a K at position 391 is substituted with D or E. Dominant negative forms of DLK as described herein are indicated using human DLK isoform 1, amino acid sequence SEQ ID NO:1, as a reference sequence. The sequence of isoform 2, as designated in Uniprot Q12852-2 is provided in SEQ ID NO:2. The isoforms differ from each other at residue 46 of the reference sequence. Isoform 1 (SEQ ID NO:1) comprises a histidine at position 46. This H residue is replaced by the sequence QCVLRDVVPLGGQGGGGPSPSPGGEPPPEPFANS in isoform 2 (SEQ ID NO:2). It will be understood that mutations described herein in relation to SEQ ID NO:1 can be introduced into SEQ ID NO:1 and/or SEQ ID NO:2. Thus, for example, a substitution K152A of SEQ ID NO:1 can be introduced into the corresponding position of SEQ ID NO:2, i.e., K at position 185 of SEQ ID NO:2 can be substituted with A.

In some embodiments, the dominant negative form of DLK comprises a substitution K152A. In some embodiments, the dominant negative form of DLK is a DLK leucine zipper domain, such as amino acids 372-487 of SEQ ID NO:1. In some embodiments, the dominant negative form of DLK comprises a substitution at position K391 and/or H393. In some embodiments, the dominant negative form of DLK comprises a substitution K391D/E and/or H393D/E. In some embodiments, when the dominant negative DLK comprises a substitution at K391 and/or H393, such as K391D/E and/or H393D/E, the DLK further comprises at least one substitution at position K398, H405, L407, V412, R414, K453, L458, or R460. In some embodiments, the substitution is K398R, H405D/E, L407D/E, V412D/E, R414D/E, K453R, L458E/E, or R460D/E.

Plasmids or vectors comprising any of the nucleic acid sequences described herein are also provided. In some embodiments, the vector is a gene therapy vector, for example, a viral vectors comprising any of the nucleic acid sequences set forth herein. Examples of viral vectors include, but are not limited to adeno-associated vectors (AAV), adenoviral vectors, lentiviral vectors, retroviral vectors, herpes simplex vectors and papilloma virus vectors. In some embodiments, the AAV is an AAV-2, an AAV-5, an AAV-8 or an AAV-9 vector. In some embodiments, the AAV vector is AAV2.7m8 and other pseudotypes, Anc80, AAV1, AAV4, AAV5, AAV6, or AAV7.

In some cases a non-biologic DLK inhibitor is administered. As discussed in the Examples below, inhibiting DLK activity reduces death of cochlear hair cells exposed to an ototoxic agent. In some cases the non-biologic DLK inhibitor is not a broad kinase inhibitor. In some cases the inhibitor preferentially inhibits activity of DLK or DLK and LZK. In some cases the inhibitor is not an amino-triazolopyridine. In some cases the inhibitor is not an-amino-pyrazolyl-[1,2,4]triazolo[1,5a]pyridine derivative). In some cases the inhibitor is 2-Amino-N-[(2S)-butan-2-yl]-7-(1-{2-methyl-1-[6-(trifluoromethyl)pyridin-3-yl]propyl}-1H-pyrazol-4-yl)[1,2,4]triazolo [1,5-α]pyridine-5-carboxamide.

In some cases a non-biologic DLK inhibitor is a 2-amino-pyrazolyl-[1,2,4]triazolo[1,5a]pyridine derivative that preferentially inhibits activity of DLK or DLK and LZK. In some embodiments, the non-biological DLK inhibitor preferentially inhibits activity of DLK or DLK and LZK and is disclosed in WO2020215094A1.

### Methods

### Evaluation of Inhibitor Function

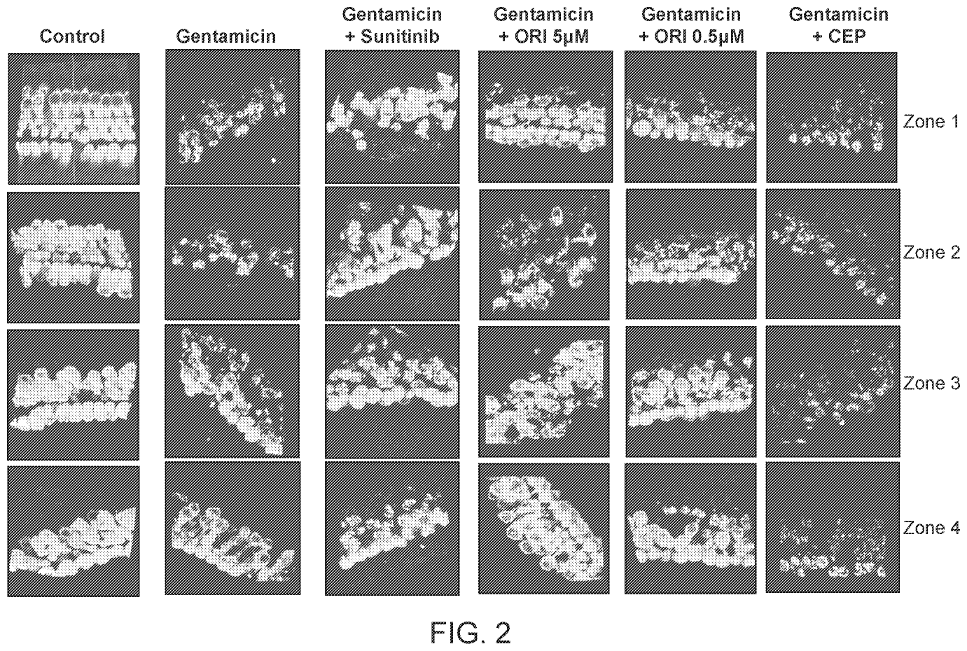

DLK inhibitor DLK inhibitor neuroprotection of hair cells and spiral ganglia can be evaluated using any number of assays. In one assay, e.g., to measure age-related and/or trauma-induced hearing loss, the function of the cochlear signal is assessed by looking at the combination of auditory brainstem responses (ABRs) and distortion product otoacoustic emissions (DPOEs) as described in Sergeyenko et al., J. Neurosciencee 33:13686-13694, 2013. Specifically, wave 1 amplitude on the ABR is measured. The graph below is from FIG. 2 of Sergeyenko et al. and is a depiction of amplitude measurement.

The selective reduction in ABRs (and not DPOEs) is evidence of inner hair cell (HC) loss, spiral ganglion cell (SGC) death and/or synaptopathy (i.e. disruption of the connection between the two). The relative contribution of each can be ascertained by histological examination of the cochlea and cell counts. Hair cells are quantified with myosin 7a staining. HC::SCN synapses is quantified by confocal imaging of cochleas stained for the post-synaptic marker, GluA2 and the presynaptic marker, CtBP2. The number of colocalizing punctae is quantified and measured at various distances down the cochlea.

In one assay, cochlear explants are used to assess inhibitor activity. Cochlear explant models are described, for example, in Wang, J. Neurosci, 2003. This model is an aminoglycoside model (neomycin); Pirvola, 2000, supra. describes a similar model.

In some embodiments, an in vitro method is employed. An illustrative assay uses an aminoglycoside-induced model (using gentamycin) of hair cell death using cochlear explants. In brief, the cochleae are collected from test animal, e.g., mice. All cochleae are combined in the same dissection maintenance bath and plated as an individual sample. The following morning, high magnification digital images of cochlea are taken and cochlear explant is assigned to groups. A desired number of explants will be used for further gentamicin treatment. Test article and positive control article are dosed ˜6 hours either prior, together with, or following to gentamicin for a duration of up to 3 days. If the culture medium is replaced (e.g. when gentamicin is added/removed), the test or positive control article will be re-administered. The cochlea are cultured and observed for health and survival. At the end of the culture cochlear explants are bathed with buffer and processed for cochleogram. Following the fixation, all the cochlear explants undergo immunofluorescent staining with anti-Myosin VIIa and Phalloidin in order to specifically label the hair cells. The explants are then mounted on microscope slides, and imaged utilizing a confocal microscope using the same laser power settings for all samples. Cochlear fragment images are then divided into 20% fragments from the apex to the base. MyosinVIIa light intensity is quantified in each fragment and compared among groups. Image analysis is performed using an imaging platform, e.g., a Fiji image analysis platform. Explants are scanned using confocal microscope with filters for 488 nm and 594 nm wave lengths. Scanning is performed using the same laser power through the entire imaging for proper light intensity calculation. For analysis, cochlea can be divided in fragment of X %. Intensity of light coming from MyoVii is quantified in each fragment and compared among groups.

Methods for introducing DLK inhibitors into a cell or a population of cells are provided herein. These methods include, in vitro, ex vivo and in vivo delivery. For example, methods for introducing a nucleic acid sequences encoding a dominant negative form of DLK into a cell are provided. Any of the nucleic acids encoding a DLK inhibitor, for example, a nucleic acid sequence encoding a dominant negative form of DLK, can be delivered or introduced into a cell via viral vector delivery, for example, via adeno-associated viral vector delivery or adenovirus vector delivery. In some embodiments, the cells are inner ear hair cells (for example, cochlear cells) and/or outer ear hair cells. In some embodiments the cells are spiral (cochlear) ganglion neurons. As set forth above, alternative gene therapy vectors include, but are not limited to, retrovirus, lentivirus, herpes simplex virus and papilloma virus vectors. Physical transfer of a nucleic acid inhibitor or a nucleic acid encoding a polypeptide inhibitor, may also be achieved by liposome-mediated transfer, direct injection (naked DNA), receptor-mediated transfer (ligand-DNA complex), electroporation, calcium phosphate precipitation or microparticle bombardment (gene gun).

### Treatment Methods

The compositions and methods provided herein can be used to treat subjects having or at risk of developing age-related hearing loss (presbycusis), hereditary hearing loss, noise-induced hearing loss, disease-associated hearing loss, exposure to toxic substances and hearing loss resulting from trauma, among other types of hearing loss. In some cases, the compositions and methods provided herein can be used to treat a subject that has Ramsay Hunt disease. In some cases, the subject has conductive hearing loss that occurs in combination with sensorineural hearing loss.

In one aspect, the disclosure further features a method of enhancing cell survival, e.g., survival of inner and outer hair cells and/or spiral (cochlear) ganglion neurons, in a subject that has hearing loss or is at risk for hearing loss, comprising administering a DLK inhibitor to the subject in an amount that reduces cell death in the cochlea of the subject compared to a normal subject that does not have hearing loss. In one embodiment, provided herein is a method for treating or preventing hearing loss in a subject comprising administering to the subject having hearing loss or at risk of developing hearing loss, a therapeutically effective amount (an amount that reduces at least partially arrests, ameliorates, delays, or prevents at least one symptom of hearing loss) of a DLK inhibitor.

In some embodiments the inhibitor is a nucleic acid sequence encoding a dual leucine zipper kinase (DLK) inhibitor. In some embodiments, the nucleic acid encodes a dominant negative form of DLK. In some embodiments, the dominant negative form of DLK comprises a substitution K152A. In some embodiments, the dominant negative form of DLK is a DLK leucine zipper domain, such as amino acids 372-487 of SEQ ID NO:1. In some embodiments, the dominant negative form of DLK comprises a substitution at position K391 and/or H393. In some embodiments, the dominant negative form of DLK comprises a substitution K391D/E and/or H393D/E. In some embodiments, when the dominant negative DLK comprises a substitution at K391 and/or H393, such as K391D/E and/or H393D/E, the DLK further comprises at least one substitution at position K398, H405, L407, V412, R414, K453, L458, or R460. In some embodiments, the substitution is K398R, H405D/E, L407D/E, V412D/E, R414D/E, K453R, L458E/E, or R460D/E. In some methods, a viral vector comprising the nucleic acid encoding the dominant negative form of DLK is administered to the subject. In some methods, the viral vector is an adeno-associated vector. In some methods, the nucleic acid sequence encoding the DLK inhibitor is administered to the inner ear hair cells and/or outer ear hair cells of the subject. In some embodiments, the DLK inhibitor is administered to spiral ganglion neurons of the subject.

In some embodiments DLK is inhibited to mitigate or prevent ototoxicity. Death of sensory hair (HC) cells or spiral ganglion cell (SG) cells are side effects of commonly used drugs such as cisplatin, carboplatin, bumetanide, and aminoglycosides (e.g., gentamicin, neomycin, kanamycin), loop diuretics and salicylates. See Ganesan et al., 2018, “Ototoxicity: A Challenge in Diagnosis and Treatment. J Audio Otol. 2018; 22(2):59-68. In some instances of hearing loss, both HC cell death and SG cell death occurs, whereas in other types of hearing loss, e.g., Ranmsey Hunt disease, only SG cell death is observed. In one aspect, the effects of ototoxic drug exposure are prevented or reduced by treatment with a DLK inhibitor. In one approach, the DLK inhibitor is administered prior to or at about the same time as treatment with an ototoxic drug. In some embodiments, the DLK inhibitor is administered within 24 hours, or within 12 hours or 6 hours, or within 4 or 2 hours of treatment with an ototoxic drug.

In some methods, administration of a DLK inhibitor improves survival of transplanted cells, e.g., transplanted stem cells or transplanted inner ear hair cells or outer ear hair cells. In some methods, administration of a DLK inhibitor treats or prevents inner ear hair cell (IHC) or outer hair cell (OHC) damage.

The compositions described herein are administered in a number of ways depending on whether local or systemic treatment is desired. The compositions are administered via any of several routes of administration, intravenously, intrathecally, intratympanically, via round window administration, via semicircular canal delivery, or via stapedotomy. In some embodiments, the compositions are administered canalostomy into the posterior semicircular canal of the subject.

When administering viral vectors, an effective amount of any of the viral vectors described herein will vary and can be determined by one of skill in the art through experimentation and/or clinical trials. For example, for in vivo injection, for example, injection directly into the inner ear of a subject, an effective dose can be from about 10⁶ to about 10¹⁵ recombinant vectors or vinons. Effective doses for any of the administration methods described herein can be extrapolated from dose-response curves derived from in vitro or animal model test systems.

The methods and compositions provided herein can be used to treat a subject having or at risk of developing any type of hearing loss. Hearing loss can be on the level of conductivity, sensorineural and/or central level. Conductive hearing loss is caused by lesions involving the external or middle ear, resulting in the destruction of the normal pathway of airborne sound amplified by the tympanic membrane and the ossicles to the inner ear fluids. Sensorineural hearing loss is caused by lesions of the cochlea or the auditory division of the eight cranial nerve. Central hearing loss is due to lesions of the central auditory pathways. In some cases, conductive hearing loss occurs in combination with sensorineural hearing loss (mixed hearing loss).

Any of the methods provided herein can further comprise administering a leucine zipper-bearing kinase (LZK) inhibitor to the subject, where the LZK inhibitor targets expression of the endogenous LZK gene, e.g., via siRNA or antisense technology; or inhibits LZK by genetically modifying the LZK gene.

In some embodiments, genetic modification to introduce a transgene encoding a DLK dominant negative polypeptide is performed using a transposase-based system for gene integration, e.g., a CRISPR/Cas-mediated gene integration, TALENS or Zinc-finger nuclease integration techniques. For example, CRISPR/Cas-mediated gene integration may be employed to introduce the DLK dominant negative polypeptide into cells in vivo or cells ex vivo, which may then be administered to a patient. In some embodiments, a gene modification system, e.g., CRISPR/Cas, TALENS or Zinc-finger nuclease integration system is employed to introduce a DLK dominant negative mutation as described herein into the endogenous gene.

In some embodiments, cells are engineered to inhibit DLK expression, e.g., using siRNA, anti-sense, other small RNA, or gene editing techniques that target endogenous DLK nucleic acid sequences. In some embodiments, cells are further engineered to inhibit LZK expression, e.g., using a second siRNA, small RNA, anti-sense or gene editing technique that specifically targets endogenous LZK nucleic acid sequences. In some embodiments, a DLK inhibitor is a micro RNA inhibitor, see, e.g., Table 3 of WO2020168111.

Any of the methods of treating hearing loss provided herein can be combined with other treatments for hearing loss, for example, a hearing aid or administration of an effective amount of a corticosteroid.

Throughout, “treat,” “treating,” and “treatment” refer to a method of reducing or delaying one or more effects or symptoms of hearing loss (e.g., trouble understanding speech, listening to television or radio at high volume, tinnitus, asking people to repeat themselves). The subject can be diagnosed with hearing loss. Treatment can also refer to a method of reducing the underlying pathology rather than just the symptoms. The effect of the administration to the subject can have the effect of, but is not limited to, reducing one or more symptoms of the disease, a reduction in the severity of the disease, the complete ablation of the disease, or a delay in the onset or worsening of one or more symptoms. For example, a disclosed method is considered to be a treatment if there is at least about a 10% reduction in hearing loss in a subject when compared to the subject prior to treatment or when compared to a control subject or control value. Thus, the reduction can be about a 10, 20, 30, 40, 50, 60, 70, 80, 90, 100%, or any amount of reduction in between. A reduction in hearing loss can also be a percentage improvement in hearing of at least about 5, 10, 20, 30, 40, 50, 60, 70, 80, 90, 100%, or any percentage in between these percentages.

As used herein, by “prevent,” “preventing,” or “prevention” is meant a method of precluding, delaying, averting, obviating, forestalling, stopping, or hindering the onset, incidence, severity, or recurrence of a disease or disorder. For example, the disclosed method is considered to be a prevention if there is a reduction or delay in onset, incidence, severity, or recurrence of hearing loss or one or more symptoms of hearing loss (e.g., trouble understanding speech, listening to television or radio at high volume, tinnitus, asking people to repeat themselves) in a subject susceptible to hearing loss compared to control subjects susceptible to hearing loss that did not receive treatment. The reduction or delay in onset, incidence, severity, or recurrence of hearing loss can be about a 10, 20, 30, 40, 50, 60, 70, 80, 90, 100%, or any amount of reduction in between.

As used throughout, by “subject” is meant an individual. The subject can be an adult subject or a pediatric subject. Pediatric subjects include subjects ranging in age from birth to eighteen years of age. Thus, pediatric subjects of less than about 10 years of age, five years of age, two years of age, one year of age, six months of age, three months of age, one month of age, one week of age or one day of age are also included as subjects. Preferably, the subject is a mammal such as a primate, and, more preferably, a human. Non-human primates are subjects as well. The term subject includes domesticated animals, such as cats, dogs, etc., livestock (for example, cattle, horses, pigs, sheep, goats, etc.) and laboratory animals (for example, ferret, chinchilla, mouse, rabbit, rat, gerbil, guinea pig, etc.). Thus, veterinary uses and medical formulations are contemplated herein.

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutations of these compounds may not be explicitly disclosed, each is specifically contemplated and described herein. For example, if a method is disclosed and discussed and a number of modifications that can be made to a number of molecules including in the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

Publications cited herein and the material for which they are cited are hereby specifically incorporated by reference in their entireties.

### EXAMPLES

### Example 1. DLK Inhibition Protects Cochlear Implants in an In Vitro Aminoglycoside Model of Hair Cell Death

Mouse cochlear explants were treated with “ORI-069” a small molecule inhibitor of DLK and LZK. ORI-069 is 2-Amino-N-[(2S)-butan-2-yl]-7-(1-{2-methyl-1-[6-(trifluoromethyl)pyridin-3-yl]propyl}-1H-pyrazol-4-yl)[1,2,4]triazolo[1,5-ca]pyridine-5-carboxamide (stereoisomers 2, eutomer). See, Example I-106 of PCT/US2020/029021, published as WO2020215094A1 (Shirok). The effect of ORI-069 was compared to treatment with a tyrosine kinase inhibitor sunitinib and a non-selective MLK inhibitor CEP-1347. CEP-1347 has been shown to increase HC survival and preserve hearing in multiple animal models (Ylikoski et al., Hearing Research 163:71-81, 2002; Pirvola et al., J Neuroscience; 20: 43-50, 2000).

Animals were euthanized in accordance with American Veterinary Medical Association guidelines and cochleae were collected. All cochleae from each group were combined in the same dissection maintenance bath and plated as a group. The cochlear explants were cultured and treated as detailed in Table 1 and Table 2, respectively, and observed for health and survival.

At the end of the culture period, the cochlear explants were bathed with 10% neutral buffered formalin (NBF) and processed for hair cell counting. Digital images of the cultures were collected before fixation.

### Imaging

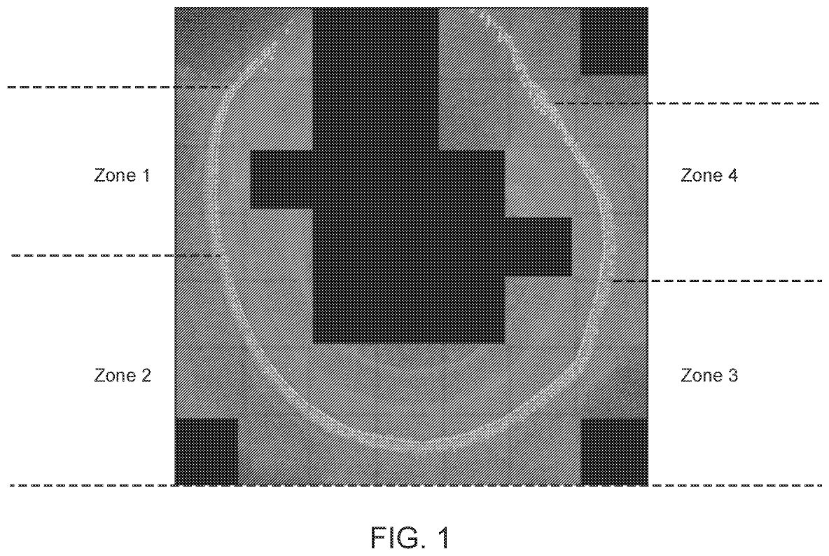

Following fixation, all cochlear explants underwent immunofluorescent staining with anti-Myosin VIIa to specifically label the hair cells. The explants were mounted on microscope slides, then imaged utilizing a confocal microscope: 20× for full scan and with 63× oil, zoom 2.25, 4 zones along the length of the cochlea: one from 50-200 μm for each extremity, one in the 33% tile and one in the 66% tile. Detailed scanning was performed using 488 nm and 594 nm filters. Zones 1, 2, 3, and 4 are shown in FIG. 1.

A blind counting of all hair cells (OHC & IHC undiscriminated) was performed on 4 to 6 zones of each explant (fragments 1, 2, 3, 4, 5 and 6) through the entire z stack generated. Counting and fragment length measure was performed by 3 different individuals. Only cells without nucleus aberration, with good staining, integrity, and well delimited cell outline were considered for counting. Cell density was determined by reporting each counting to the length of the fragment. Image analysis was performed using Fiji image analysis platform, a distribution of ImageJ.

TABLE 1
Cochlear Explant Treatment Matrix

| | | Number Cochlear | | | |
|---|---|---|---|---|---|
| | | Explants | | Gentamicin | |
| Group | Groups | Excised | Treated | (50 μM) | Treatment |
| 1 | Control | 6 | No | No | None |
| 2 | Control Gentamycin 50 μM | 6 | No | Yes | None |
| 3 | Positive Control #1 | 6 | Yes | Yes | CEP-1347 |
| | Gentamycin + CEP-1347 (0.5 μM) | | | | 0.5 μM |
| 4 | Positive Control #2 | 6 | Yes | Yes | Sunitinib |
| | Gentamycin + Sunitinib (0.5 μM) | | | | 0.5 μM |
| 5 | Gentamycin + ORI-069 Dose 1 | 6 | Yes | Yes | ORI-069 |
| | (0.5 μM) | | | | 0.5 μM |
| 6 | Gentamycin + ORI-069 Dose 2 | 6 | Yes | Yes | ORI-069 |
| | (5 μM) | | | | 5 μM |

| | |
|---|---|
| TOTAL | 36 |

TABLE 2
Treatment Protocol

| | | Route of | Volume | Treatment | Dose or |
|---|---|---|---|---|---|
| Identification | Formulation | Administration | Administered | Regimen | Concentration |
| ORI-069 | Culture | Culture | Up to 200 μL | 3 days | 0.5 μM & 5 μM |
| | medium | medium | | | |
| CEP-1347 | Culture | Culture | Up to 200 μL | 3 days | 0.5 μM |
| | medium | medium | | | |
| Sunitinib | Culture | Culture | Up to 200 μL | 3 days | 0.5 μM |
| | medium | medium | | | |
| Gentamycin | Culture | Culture | Up to 200 μL | Single (16 h) | 50 μM |
| | medium | medium | | | |

### Results

FIG. 2 shows 3-D Z-stack reconstructions for control and treated explants. The results indicate that treatment with ORI-069 protected explant cultures.

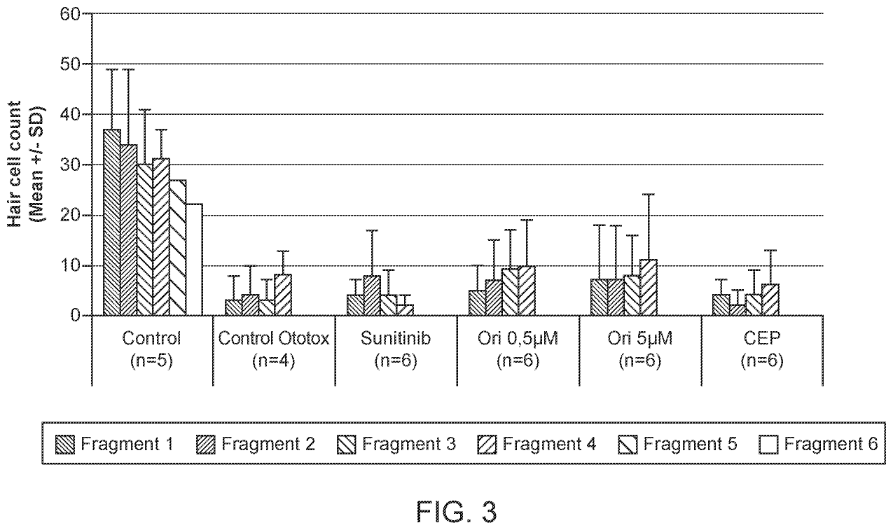

Cochlear explant hair cell count results for six fragments (zones) are shown in FIG. 3. Viable hair cells were counted in all fragments of explants. In the untreated explants (control group), the presence of 17-52 viable hair cells were counted in each fragment. The number of viable hair cells was very low in gentamycin (0-14 hair cells/fragment) compared to untreated explants. In gentamycin treated explants co-administered with the different treatments, the number of viable hair cells was also lower compared to untreated explants: Sunitinib (0-18 hair cells/fragment), ORI-069 0.5 μM (0-20 hair cells/fragment), ORI-069 5 μM (0-32 hair cells/fragment) or CEP-1347 (0-18 hair cells/fragment) treated explants.

In the ORI-069 5 μM (0-32 hair cells/fragment) treated group, the number of viable hair cells was lower compared to untreated explants (17-52 hair cells/fragment), but the number of hair cells was higher compared to gentamycin treated explants (0-14 hair cells/fragment). The explants treated with Sunitinib or CEP-1347 presented the same low level of hair cells as the gentamycin treated explants. The high dosage treatment group of ORI-069 (5 μM) trended higher compared to the other gentamycin treatment groups. In this particular assay, positive control (CEP-1347) did not show a protective effect, which contradicts previous studies (Bodmer, et al., Laryngorhinootologie 81(12):853-6, 2002; Pirvola, et al., J Neurosci. 20(1):43-50, 2000; Ylikoski, et al., Hear Res. 166(1-2):33-43, 2002.

These experiments indicate that inhibition of DLK and LZK protects cochlear hair cells from the effects of ototoxic drugs.

Illustrative sequences
Human DLK polypeptide sequence, isoform 1,
UniProt Q12852-1
SEQ ID NO: 1
MACLHETRTP SPSFGGFVST LSEASMRKID PDTSDCTPEK
DITPTHVIQL HEQDAGGPGG AAGSPESRAS RVRADEVRIQ
CQSGSGFLEG LEGCLRPVWT MIGKAYSTEH KQQQEDIWEV
PFEBILDLQW VGSGAQGAVE IGREHGEEVA VKKVRDIKET
DIKHIRKLKH PNIITFKGVC TQAPCYCILM EFCAQGQLYE
VLRAGRPVTP SLIVDWSMGI AGGMNYLHLH KIIHRDLKSP
NMLITYDDVV KISDEGTSKE ISDKSTKMSF AGTVAWMAPE
VIRNEPVSEK VDIWSFGVVI WELLTGEIPY KDVDSSAIIW
GVGSNSLHIP VPSSCPDGFK ILLRQCWNSK PRNRPSFRQI
LLHLDIASAD VISTPQETYF KSQAEWREEV KIHFEKIKSE
GTCLHRLEEE LVMRRREELR HALDIREHYE RKLERANNLY
MELNALMLQL ELKERELLRR EQALERRCPG LLKPHPSRGL
LHGNTMEKLI KKRNVPQKLS PHSKRPDILK TESLLPKLDA
ALSGVGLPGC PKGPPSPGRS RRGKTRHRKA SAKGSCGDLP
GLRTAVPPHE PGGPGSPGGL GGGPSAWEAC PPALRGLHHD
ILLRKMSSSS PDLLSAALGS RGRGATGGAG DPGSPPPARG
DIPPSEGSAP GSTSPDSPGG AKGEPPPPVG PGEGVGLIGT
GREGTSGRGG SRAGSQHLTP AALLYRAAVT RSQKRGISSE
EEEGEVDSEV ELTSSQRWPQ SINMRQSLST ESSENPSDGE
EGTASEPSPS GTPEVGSTNT DERPDERSDD MCSQGSEIPL
DPPPSEVIPG PEPSSLPIPH QELLRERGPP NSEDSDCDST
ELDNSNSVDA IRPPASLPP
Human DLK isoform 2 UniProt Q12852
SEQ ID NO: 2
MACLHETRTP SPSFGGFVST LSEASMRKLD PDTSDCTPEK
DLTPTQCVLR DVVPLGGQGG GGPSPSPGGE PPPEPFANSV
LQLHEQDAGG PGGAAGSPES RASRVRADEV RLQCQSGSGF
LEGLFGCLRP VWTMIGKAYS TEHKQQQEDL WEVPFEEILD
LQWVGSGAQG AVELGRFHGE EVAVKKVRDL KETDIKHLRK
LKHPNIITFK GVCTQAPCYC ILMEFCAQGQ LYEVLRAGRP
VTPSLLVDWS MGIAGGMNYL HLHKIIHRDL KSPNMLITYD
DVVKISDEGT SKELSDKSTK MSFAGTVAWM APEVIRNEPV
SEKVDIWSFG VVLWELLTGE IPYKDVDSSA IIWGVGSNSL
HLPVPSSCPD GFKILLRQCW NSKPRNRPSF RQILLHLDIA
SADVLSTPQE TYFKSQAEWR EEVKLHFEKI KSEGTCLHRL
EEELVMRRRE ELRHALDIRE HYERKLERAN NLYMELNALM
LQLELKEREL LRREQALERR CPGLLKPHPS RGLLHGNTME
KLIKKRNVPQ KLSPHSKRPD ILKTESLLPK LDAALSGVGL
PGCPKGPPSP GRSRRGKTRH RKASAKGSCG DLPGLRTAVP
PHEPGGPGSP GGLGGGPSAW EACPPALRGL HHDLLLRKMS
SSSPDLLSAA LGSRGRGATG GAGDPGSPPP ARGDTPPSEG
SAPGSTSPDS PGGAKGEPPP PVGPGEGVGL LGTGREGTSG
RGGSRAGSQH LTPAALLYRA AVTRSQKRGI SSEEEEGEVD
SEVELTSSQR WPQSLNMRQS LSTESSENPS DGEEGTASEP
SPSGTPEVGS TNTDERPDER SDDMCSQGSE IPLDPPPSEV
IPGPEPSSLP IPHQELLRER GPPNSEDSDC DSTELDNSNS
VDALRPPASL PP

1. A method for treating or preventing hearing loss in a subject comprising administering to the subject having hearing loss or at risk of developing hearing loss, a therapeutically effective amount a dual leucine zipper kinase (DLK) inhibitor.

2. The method of claim 1 wherein the hearing loss is selected from the group consisting of age-related hearing loss, hereditary hearing loss, chemotherapy induced hearing loss, aminoglycoside-induced hearing loss, trauma-induced hearing loss, disease-induced hearing loss, and noise-induced hearing loss.

3. The method of claim 1, wherein the hearing loss is the result of loss of auditory hair cells (HC).

4. The method of claim 1, wherein the hearing loss is the result of loss of spiral ganglion cells (SG).

5. The method of claim 2, wherein the subject has hearing loss following treatment with an ototoxic drug.

6. The method of claim 2, wherein the DLK inhibitor is administered to a subject prior to treatment with an ototoxic drug.

7. The method of claim 5, wherein the ototoxic drug is an aminoglycoside.

8. The method of claim 1, wherein the DLK inhibitor is a dominant negative form of a DLK polypeptide.

9. The method of claim 8 wherein a nucleic acid sequence encoding the dominant negative form of the DLK polypeptide is administered to the subject.

10. The method of claim 8, wherein the dominant negative form of the DLK polypeptide comprises a substitution K152A; is a DLK leucine zipper domain amino acids 372-487 of SEQ ID NO: 1; or comprises a substitution K391D/E and/or H393D/E.

11. The method of claim 10, wherein, when the dominant negative form of the DLK polypeptide comprises a substitution K391D/E and/or H393D/E, the dominant negative form further comprises at least one substitution K398R, H405D/E, L407D/E, V412D/E, R414D/E, K453R, L458E/E, or R460D/E.

12. The method of claim 9, wherein a viral vector comprising the nucleic acid encoding the dominant negative form of the DLK polypeptide is administered to the subject.

13. The method of claim 12, wherein the viral vector is an adeno-associated vector.

14. The method of claim 1, wherein the inhibitor is administered to Inner Ear Hair Cells and/or Outer Ear Hair Cells of the subject.

15. The method of claim 1, wherein the inhibitor is administered to the spiral ganglion in the cochlea of the subject.

16. The method of claim 1, wherein the inhibitor or viral vector is administered intravenously, intrathecally, intratympanically, via round window administration, via semicircular canal delivery, or via stapedotomy.
